(19) United States
(12) Patent Application Publication
(10) Pub. No.: US 2025/0008996 A1
(43) Pub. Date: Jan. 9, 2025

(54) Flavored Food and Beverage Products

(71) Applicants: Firmenich SA, Satigny (CH); Healthtech Bio Actives S.L.U., Barcelona (ES)

(72) Inventors: Francisco Javier Crespo, Barcelona (ES); Tom Nelly Aime D'Hoore, Barcelona (ES); Ronald H. Skiff, Plainsboro, NJ (US); Long In Lou, Plainsboro, NJ (US); Maxime Delattre, Satigny (CH)

(73) Assignees: Firmenich SA, Satigny (CH); Healthtech Bio Actives S.L.U., Barcelona (ES)

(21) Appl. No.: 18/892,825

(22) Filed: Sep. 23, 2024

Related U.S. Application Data
(63) Continuation of application No. 15/309,031, filed on Nov. 4, 2016, now abandoned.
(60) Provisional application No. 61/988,270, filed on May 4, 2014.

(51) Int. Cl. A23L 27/20; A23L 2/60; A23L 27/00; A23L 27/30; A23L 33/10; C07D 311/32

(52) U.S. Cl. CPC A23L 27/2052; A23L 2/60; A23L 27/30; A23L 27/36; A23L 27/88; A23L 33/10; C07D 311/32; A23V 2002/00

(57) ABSTRACT

A method of enhancing the sweetness of sweetener in a food or beverage product comprising adding Naringenin to the product in an amount of 50 ppm to 200 ppm by weight of the total weight of the product wherein the naringenin does not block the bitter taste of the product when compared to the beverage without naringenin. Also provided here is a food or beverage product comprising naringenin in an amount of from 50 to 200 ppm, by weight, of the total weight of the product, and a sweetener wherein the product is not a product selected from coffee, tea, a cosmetic and a pharmaceutical.

### FIELD

The inventions described herein have use in foods and beverages, particularly those that rely on sweeteners and other flavor components and more particularly those that might rely on compounds and ingredients found in nature.

### BACKGROUND

Naringenin (2,3-Dihydro-5,7-dihydroxy-2(-4-hydroxyphenyl)-4H-1-bcmzopyran-4-one) has been reported for use as an antioxidant and therapeutic for example in dietary supplements, in tea, in a cosmetic formulation and in a pharmaceutical product. Naringenin has further been reported for example as a bitter blocker in coffee.

SUMMARY

Provided herein is a method of enhancing the sweetness of a sweetener in a food or beverage product comprising adding Naringenin to the product in an amount of 30 ppm to 200 ppm by weight of the total weight of the product wherein the Naringenin does not block the bitter taste of the product when compared to the beverage without Naringenin.

Also provided herein is a food or beverage product comprising Naringenin in an amount of from 30 to 200 ppm, by weight, of the total weight of the product, and a sweetener wherein the product is not a product selected from coffee, tea, a cosmetic and a pharmaceutical.

### BRIEF DESCRIPTION OF THE DRAWINGS

FIG. 1 shows the effects of 125 ppm Naringenin in sucrose sweetened water.

FIG. 2 shows the effects of 175 ppm Naringeninin sucrose sweetened water.

FIG. 3 shows the effects of 175 ppm Naringeninin in a sucrose sweetened sample.

FIG. 4 shows the effects of 125 ppm Naringenin in a sucrose sweetened sample

FIG. 5 shows the effects of 125 ppm and 150 ppm Naringenin in a high fructose corn syrup (HFCS) solution.

FIG. 6 shows the effects of 10, 50, 100 and 200 ppm Naringenin in a sucrose solution.

FIG. 7 shows the effects of 50, 100 and 150 ppm Naringenin in a sucrose solution.

FIG. 8 shows the effects of 100 ppm Naringenin in an orange juice based soft drink.

FIG. 9 shows the effects of 10, 25 and 50 ppm Naringenin in a Reb A sweetened sample.

FIG. 10 shows the effects of 10, 25, 50 ppm, 75. 100 and 150 ppm Naringenin in a Reb A sweetened sample

FIG. 11 shows the effects of 10, 25, 50 ppm, 75. 100 and 150 ppm Naringenin in a SG95 (stevia glycosides) sweetened sample

FIG. 11 shows the effects of 10, 25, 50 ppm, 75. 100 and 150 ppm Naringenin in a SG95 (stevia glycosides) sweetened samplc.

FIG. 13 shows the effects of Naringenin in stevia sweetened lemon flavored bottled water.

FIG. 14 shows the effects of Naringenin in stevia sweetened strawberry flavored bottled water.

### DETAILED DESCRIPTION

For the descriptions herein and the appended claims, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

In one embodiment, Naringenin is provided in a food or beverage in an amount of 50 ppm to 200 ppm by weight of the total weight of the beverage, particularly in combination with sucrsose or fructose more particularly in combination with 5% sucrose or fructose, by weight of the total weight of the food or beverage.

In one embodiment, Naringenin is provided in a food or beverage in an amount of 75 ppm to 200 ppm by weight of the total weight of the beverage.

In a further embodiment, Naringenin is provided in a food or beverage in an amount of 125 ppm to 200 ppm by weight of the total weight of the beverage.

In another embodiment, Naringenin is provided in a food or beverage in an amount of 125 to ppm to 175 ppm by weight of the total weight of the beverage

In another embodiment, Naringenin is provided in a food or beverage in an amount of 30 ppm to 175 ppm by weight of the total weight of the beverage.

In another embodiment, Naringenin is provided in a food or beverage in an amount of 30 ppm to 100 ppm by weight of the total weight of the beverage.

In another embodiment, Naringenin is provided in a food or beverage in an amount of 30 ppm to 75 ppm by weight of the total weight of the beverage.

In another embodiment, Naringenin is provided in a food or beverage in an amount of 30 ppm to 50 ppm by weight of the total weight of the beverage.

It has been discovered that Naringenin, when tasted in water or in a model beverage solution of water and/or citric acid, it does not contain (for the most part) sweetness on its own. However, when combined with others sweeteners, Naringenin provides sweetness enhancement to composition comprising a sweetener. In some embodiments, Naringenin provides a sweetness equivalent to 1-2° brix of sucrose equivalents. Unexpectedly, it was found that Naringenin did not reduce the bitterness of some of the formulations tested herein. Further, it was found in one formulation that Naringenin actually added bitterness.

In another embodiment, the methods and compositions provided herein can be provided in the substantial absence of a bitter substance, that is provided in an amount below, where the bitter substance confers a bitter taste to a product or beverage. In a particular embodiment, the compositions and methods provided herein are provided in the absence of a bitter substance. A bitter substance may be selected from the group consisting of xanthine alkaloids (for example caffeine, theobromine), quinoline derivatives (for example quinine), polyphenols (for example catechols, flavonols, y-oryzanol, hesperitin), pharmaceutically active compounds (for example fluoroquinolone antibiotics, aspirin, ˜-lactam antibiotics, ambroxol, paracetamol, aspirin, guaifenesin), denatonium benzoate, sucralose octaacetate, potassiumchloride, magnesium salts, urea, bitter amino acids (for example tryptophan) and bitter peptide fragments (for example having a terminal leucine or isoleucine radical).

In another embodiment, Naringenin enhances the flavor profile of a food or beverage, particularly candied intensity and herbal intensity in orange and strawberry flavor bottled water.

In another embodiment, Naringenin enhances the overall mouth-feel of a food or beverage.

In another embodiment, a food or beverage provided herein is not a tea or coffee product. more particularly the food or beverage provided herein is not a coffee food or beverage.

In another embodiment, the sweetener provided herein is selected from the group consisting common saccharide sweeteners, e.g., sucrose, fructose (e.g., D-fructose), glucose (e.g., D-glucose); sweetener compositions comprising natural sugars, such as corn syrup (including high fructose corn syrup) or other syrups or sweetener concentrates derived from natural fruit and vegetable sources; semisynthetic “sugar alcohol” sweeteners such as erythritol, isomalt, lactitol, mannitol, sorbitol, xylitol, maltodextrin, glycerol, threitol, arabitol, ribitol, and dulcitol; artificial sweeteners such as miraculin, aspartame, superaspartame, saccharin, saccharin-sodium salt, acesulfame-K, cyclamate, sodium cyclamato, and alitame; other sweeteners such as trchalosc, melizitose, melibiose, raffinose, palatinosc, lactulose, cyclamic acid, mogroside, tagatose (e.g., D-tagatose), maltose, galactose (e.g., D-galactose), L-rhamnose, D-sorbose, maunose (e.g., D-maunose), lactose, L-arabinose,D-ribose, D-glyceraldehyde, curculin, brazzein, mogroside, Neohesperidin dihydrochalcone (NHDC), neotame and other aspartame derivatives, D-tryptophan, D-leucine, D-threonine, glycine, D-asparagine, D-phenylalanine, L-proline, maltitol, hydrogenated glucose syrup (HGS), magap, sucralose, lugduname, sucrononate, sucrooctate, monatin, phyllodulcin, hydrogenated starch hydrolyzate (HSH), stevioside, rebaudioside A, rebaudioside D, rebadioside M, and other sweet Stevia based glycosides, lo han guo, thaumatin, monellin, carrelameand amd other guanidine-based sweeteners. Particularly, the sweetener is a high potency sweetener, particularly it is selected from the group consisting of saccharin, aspartame, cyclamate, sucralose, saccharine, stevia, rebauasdioside A, neotame, acesulfame K, suerose, glucose fructose and sorbitol, more particularly it is selected from the group consisting of fructose and stevia.

In a further embodiment the sweetener is selected from the group consisting of sucrose, fructose, and stevia. In a further embodiment, the sweetener comprises a purified stevia extract having a high purity combination of nine sweet steviol glycosides found within the stevia leaf. The purified stevia extract may be represented by high purity combination of nine sweet steviol glycosides found within the stevia leaf wherein Reb A accounts for over half of the final composition (e.g., SG95 sold by PureCircle).

In one embodiment, Naringenin significantly increases the sweetness intensity of a lemon flavored bottled water containing 300 ppm of Steviol Glycosides (SG 95 from PureCircle) without significantly impacting any of the other flavor attributes. Moreover, the addition of 125 ppm Naringenin can maintain the sweetness intensity of a lemon flavored beverage having a 50 ppm reduced level of SG95. No maksing of bitterness was observed.

In another embodiment, 125 ppm Naringenin significantly increases the sweetness of 375 ppm Steviol Glycosides (SG95 from PureCircle) for example in a Strawberry flavored bottled water while also increasing the overall mouth-feel intensity, and lingering sweetness intensity. Moreover, the addition of 150 ppm Naringenin allows one to maintain the sweetness intensity of the beverage while reducing the level of Steviol glycosides (SG95). This allows one to create a beverage with reduced lingering sweetness and licorice aftertaste, providing a more pleasant tasting beverage. No masking of bitterness was observed.

In another embodiment, the addition from 10 to 25 ppm Naringenin to 150 ppm Reb.A solution does not bring any sweetness. In another embodiment, the addition of 50 ppm Naringenin to 150 ppm Reb.A solution enhances the sweetness intensity to 4% sucrose level.

In a further embodiment, the addition from 10 to 75 ppm Naringenin to a 300 ppm Reb.A solution has a 17% of sweetness enhancement effect. In another aspect, the addition of 100 and 150 ppm Naringenin to 300 ppm Reb.A solution has a small loss of sweetness (8%). In one embodiment provided herein is a food or beverage comprising from about 10 to 75 ppm Naringenin to up about 300 ppm Reb A.

In one embodiment, the addition of 10-25 ppm Naringenin to 150 ppm steviol glycosides (e.g., SG95) doesn't have any effect on sweetness. In another embodiment, the addition from from 50 to 150 ppm Naringenin to 150 ppm steviol glycosides (e.g., SG95) solution has a sweetness enhancement effect.

In another embodiment, the addition of 10-25 ppm Naringenin to 300 ppm steviol glycosides (e.g., PureCircle SG95) appears not to have any effect on sweetness. On the other hand, the addition of 50 to 150 ppm Naringenin to 300 ppm SG95 solution has a sweetness enhancement effect.

The compositions and methods provided herein have use in food or beverage products. When the food product is a particulate or powdery food, the dry particles may easily be added thereto by dry-mixing. Typical food products are selected from the group consisting of an instant soup or sauce, a breakfast cereal, a powdered milk, a baby food, a powdered drink, a powdered chocolate drink, a spread, a powdered cereal drink, a chewing gum, an effervescent tablet, a cereal bar, and a chocolate bar. The powdered foods or drinks may be intended to be consumed after reconstitution of the product with water, milk and/or a juice, or another aqueous liquid.

The food product may be selected from the group consisting of condiments, baked goods, powdery food, bakery filings and Fluid dairy products.

Condiments include, without limitation, ketchup, mayonnaise, salad dressing, Worcestershire sauce, fruit-flavored sauce, chocolate sauce, tomato sauce, chili sauce, and mustard.

Baked goods include, without limitation, cakes, cookies, pastries, breads, donuts and the like.

Bakery fillings include, without limitation, low or neutral pH fillings, high, medium or low solids fillings, fruit or milk based (pudding type or mousse type) fillings, hot or cold make-up fillings and nonfat to full-fat fillings.

Fluid dairy products include, without limitation, non-frozen, partially frozen and frozen fluid dairy products such as, for example, milks, ice creams, sorbets and yogurts.

Beverage products include, without limitation, carbonated soft drinks, including cola, lemon-lime, root beer, heavy citrus (“dew type”), fruit flavored and cream sodas; powdered soft drinks, as well as liquid concentrates such as fountain syrups and cordials; coffee and coffee-based drinks, coffee substitutes and cereal-based beverages; teas, including dry mix products as well as ready-to-drink teas (herbal and tealeaf based); fruit and vegetable juices and juice flavored beverages as well as juice drinks, nectars, concentrates, punches and “ades”; sweetened and flavored waters, both carbonated and still; sport/energy/health drinks; alcoholic beverages plus alcohol-free and other low-alcohol products including beer and malt beverages, cider, and wines (still. sparkling, fortified wines and wine coolers); other beverages processed with heating (infusions, pasteurization, ultra high temperature, ohmic heating or commercial aseptic sterilization) and hot-filled packaging; and cold-filled products made through filtration or other preservation techniques. SG95 is a natural, high purity combination of nine sweet steviol glycosides found within the stevia leaf. Reb A accounts for over half of the final composition.

The below examples are illustrative only and are not meant to limit the claims or embodiments described herein.

### EXAMPLES

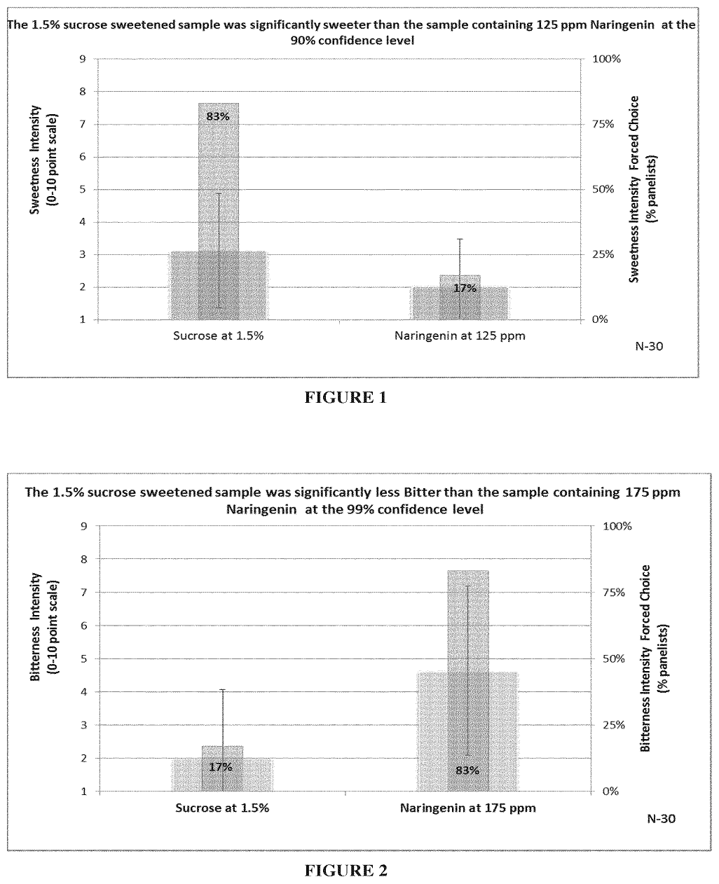

Example 1. Two samples were prepared. The first sample comprised Naringenin at 125 ppm in water. The second sample comprised Sucrose at 1.5% in water. Both samples were tested for sweetness intensity by thirty participants. The data displayed in FIG. 1 shows that Naringenin is essentially not sweet on its own.

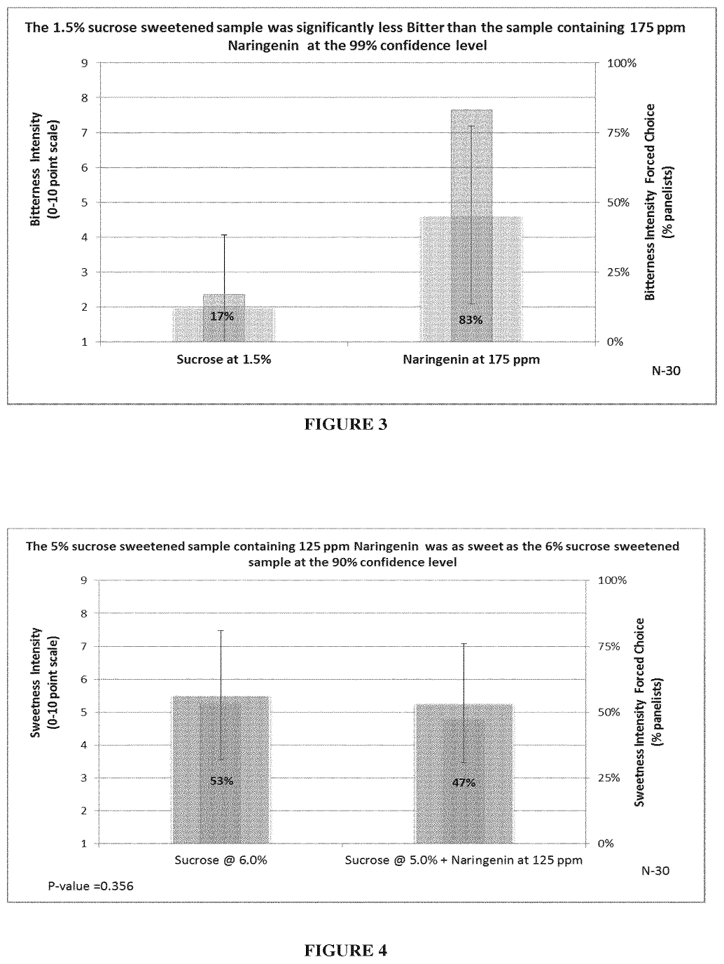

The test was repeated (thirty participants) with 175 ppm Naringenin and again the data show that Naringenin is essentially not sweet on its own (FIG. 2) at this concentration. This sample was also tested for bitterness and the data displayed in FIG. 3 show that Naringenin is bitter at 175 ppm as compared to the sucrose sample.

Example 2. Two samples were prepared. The first sample was a 6% sucrose solution in water. The second sample was a 5% sucrose solution in water with 125 ppm Naringenin. The samples were tested for sweetness intensity by thirty participants. The data displayed in FIG. 4 show that the sample with Naringenin enhanced the sweetness of the 5% solution to about the same level of sweetness as the 6% sucrose solution without Naringenin.

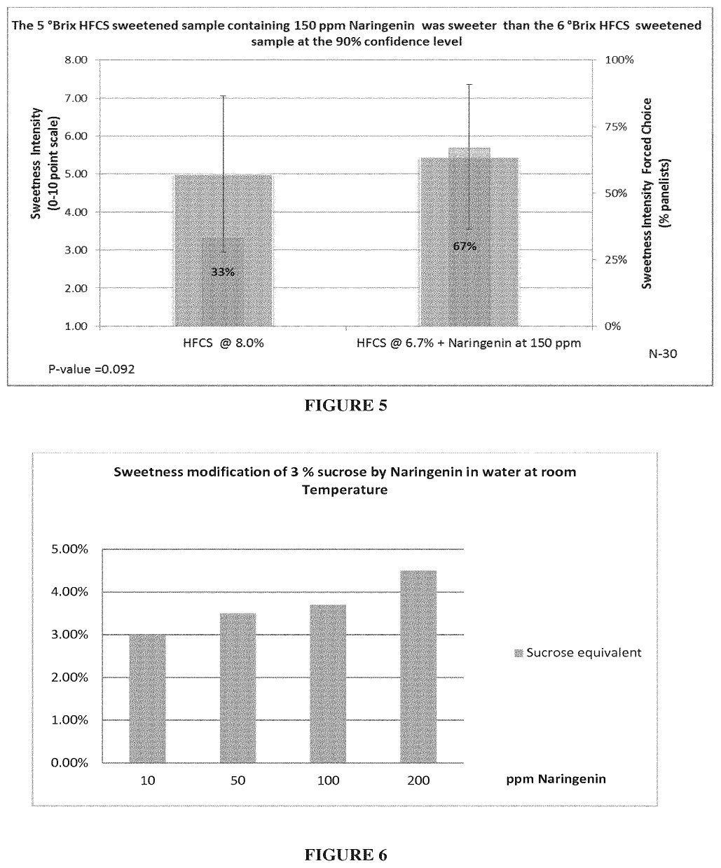

Example 3. Two samples were prepared. The first sample was a 6° Brix high fructose corn syrup (HFCS) solution. The second sample was a 5° Brix HFCS with 125 ppm Naringenin. The samples were tested for sweetness intensity by thirty participants. The data displayed in FIG. 5 show that the sample with the Naringenin and 5° Brix HFCS had essentially the same sweetness intensity as the sample without the Naringenin.

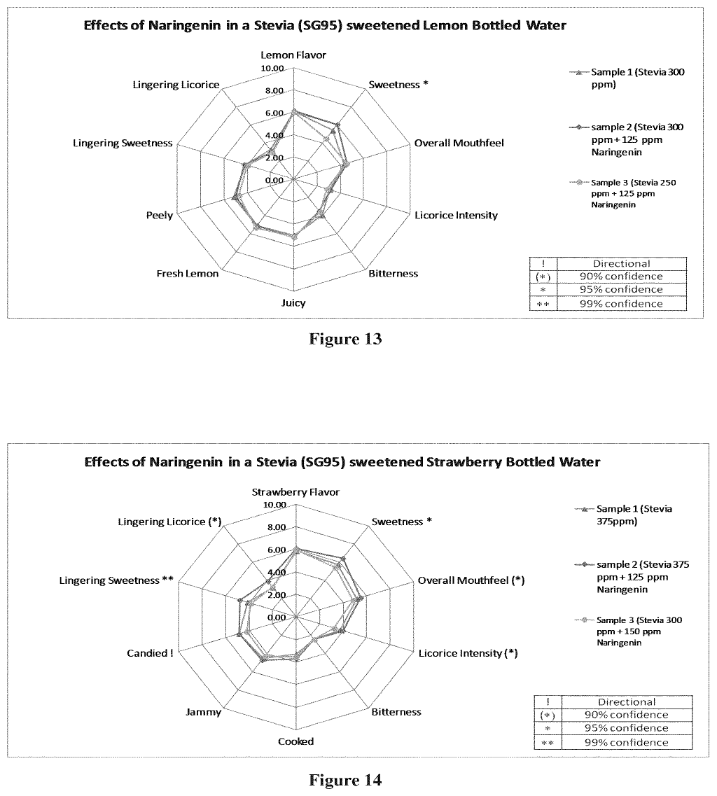

Example 4. Three samples were prepared. The first sample was a 3 ppm purified stevia extract (SG 95 from PureCircle) I lemon flavored bottled water. The second sample contained 300 ppm SG 95 and 125 ppm Naringenin in lemon flavored bottled water. The third sample contained 250 ppm SG 95 and 125 ppm Naringenin in lemon flavored bottled water. The samples were tested by thirty-one participants for lemon flavor, sweetness, overall mouth-feel, licorice intensity, bitterness, fresh lemon, peely, lingering sweetness and lingering licorice. The data as displayed in FIG. 13 show that Naringenin significantly increases the sweetness intensity of a lemon flavored bottled water containing 300 ppm of Steviol Glycosides (SG 95 from PureCircle) without significantly impacting any of the other flavor attributes. Moreover, example 4 shows that the addition of 125 ppm Naringenin can maintain the sweetness intensity of a lemon flavored beverage having a 50 ppm reduced level of SG95. No masking of bitterness was observed.

Example 5. Three samples were prepared. The first sample was a strawberry flavored bottled water sample containing 375 ppm SG 95. The second sample was a strawberry flavored bottled water containing 375 ppm SG 95 plus 125 ppm Naringenin. The third sample was a strawberry flavored bottled water sample containing 300 ppm SG 95 and 150 ppm Naringenin. The samples were tested by thirty participants for strawberry flavor, sweetness, overall mouth-feel, licorice intensity, bitterness, cooked, jammy, candied, lingering sweetness and lingering licorice. The data displayed in FIG. 14 show that 125 ppm Naringenin significantly increases the sweetness of 375 ppm Steviol Glycoside (SG95 from PureCirele) in a Strawberry flavored bottled water while also increasing the overall mouth-feel intensity, and lingering sweetness intensity. Moreover, the addition of 150 ppm Naringenin allows one to maintain the sweetness intensity of the beverage while reducing the level of Steviol glycosides (SG95). This allows one to create a beverage with reduced lingering sweetness and licorice aftertaste, providing a more pleasant tasting beverage. No masking of bitterness was observed.

Example 6. Four samples were prepared and compared with a 3% sucrose solution in water. The first sample comprised sucrose at 3% with 10 ppm Naringenin (Interquim, S.A). The second sample was a 3% sucrose solution with 50 ppm Naringenin. The third sample was a 3% sucrose solution with 100 ppm Naringenin. The fourth sample was a 3% sucrose solution with 200 ppm Naringenin. All samples were tested for sweetness intensity at room temperature by four experts especially trained to evaluate sweet products. The data displayed in FIG. 6 shows that the addition from 50 to 200 ppm Naringenin to 3% sucrose solution enhances the sweetness intensity of 3% sucrose in water.

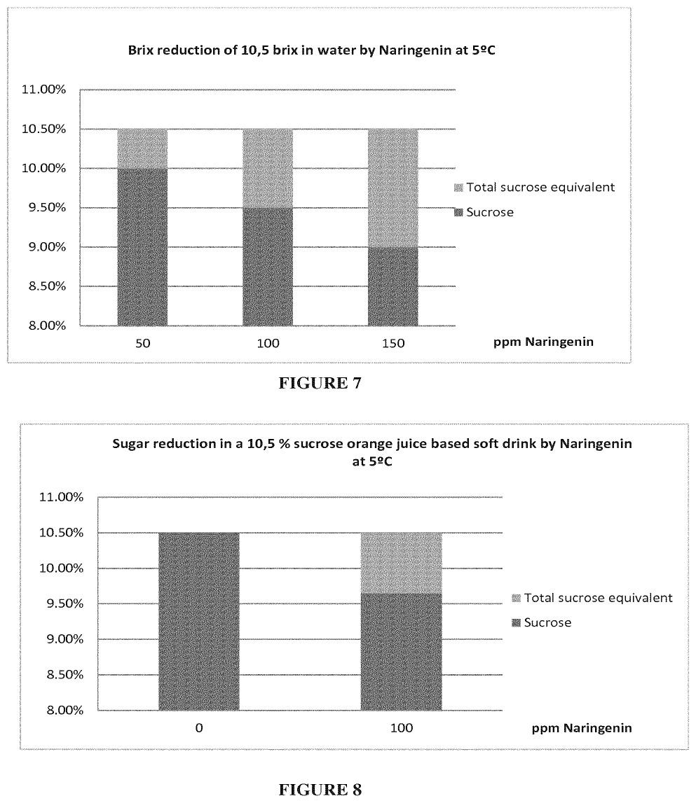

Example 7: Three samples were prepared and compared with a 10.5% sucrose solution in water. The first sample comprised sucrose at 10% with 50 ppm Naringenin. The second sample was a 9.5% sucrose solution with 100 ppm Naringenin. The third sample was a 9% sucrose solution with 150 ppm Naringenin. All samples were tested for sweetness intensity at the refrigeration Temperature by four experts especially trained to evaluate sweet products. The data displayed in FIG. 7 shows that the addition from 50 to 150 ppm of Naringenin to 10, 9.5 and 9% sucrose solutions in water maintains the same sweetness intensity level as a 10.5% sucrose solution.

Example 8. Two samples were prepared. The first sample was a 10.5% sucrose orange juice based soft drink. The second sample was a 9.65% sucrose orange juice based SD with 100 ppm Naringenin. The samples were tested for sweetness intensity at refrigeration Temperature by four experts especially trained to evaluate sweet products. The data displayed in FIG. 8 shows that the sample with the Naringenin and 9.65% sucrose had essentially the same sweetness intensity as the sample with 10.5% sucrose.

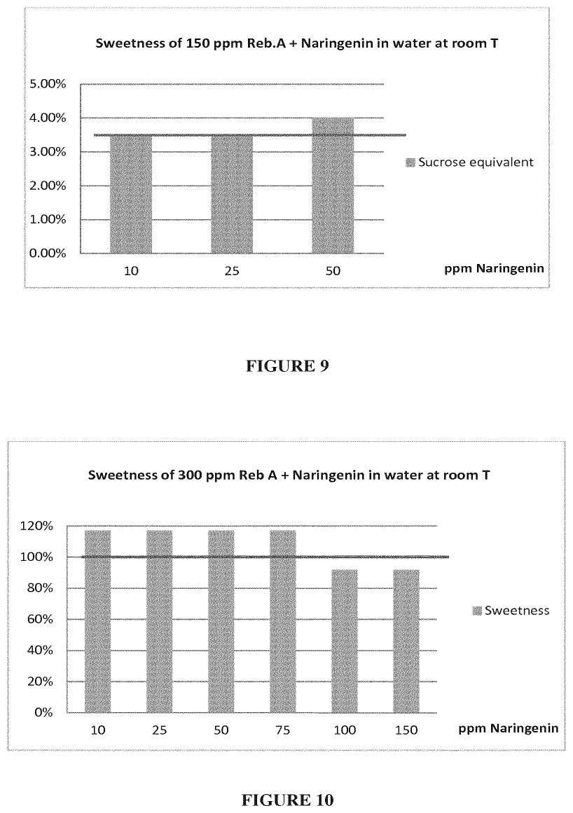

Example 9: Three samples were prepared and compared with 150 ppm Reb.A (PureCircle) in water; equisweet to 3.5% sucrose (in red, FIG. 9). The first sample comprised 150 ppm Reb.A with 10 ppm Naringenin. The second sample was a 150 ppm Reb.A with 25 ppm Naringenin. The third sample comprised 150 ppm Reb.A with 50 ppm Naringenin. All samples were tested for sweetness intensity at room Temperature by four experts especially trained to evaluate sweet products. The data displayed in FIG. 9 shows that the addition from 10 to 25 ppm Naringenin to 150 ppm Reb.A solution doesn't bring any sweetness. On the other hand, the addition of 50 ppm Naringenin to 150 ppm Reb.A solution enhances the sweetness intensity to 4% sucrose level.

Example 10: Six samples were prepared and compared with 300 ppm Reb.A in water; 100% sweetness level (in red, FIG. 10). The first sample comprised 300 ppm Reb. A with 10 ppm Naringenin. The second sample was a 300 ppm Reb.A with 25 ppm Naringenin. The third sample was a 300 ppm Reb.A with 50 ppm Naringenin. The fourth sample comprised 300 ppm Reb.A with 75 ppm Naringenin. The fifth sample was a 300 ppm Reb.A with 100 ppm Naringenin. The sixth sample was a 300 ppm Reb.A with 150 ppm Naringenin. All samples were tested for sweetness intensity at room Temperature by four experts especially trained to evaluate sweet products. The data displayed in FIG. 10 shows that the addition from 10 to 75 ppm Naringenin to 300 ppm Reb.A solution has a 17% of sweetness enhancement effect; all of them were equisweet to 350 ppm Reb.A in water. On the other hand, the addition of 100 and 150 ppm Naringenin to 300 ppm Reb.A solution has a small loss of sweetness (8%).

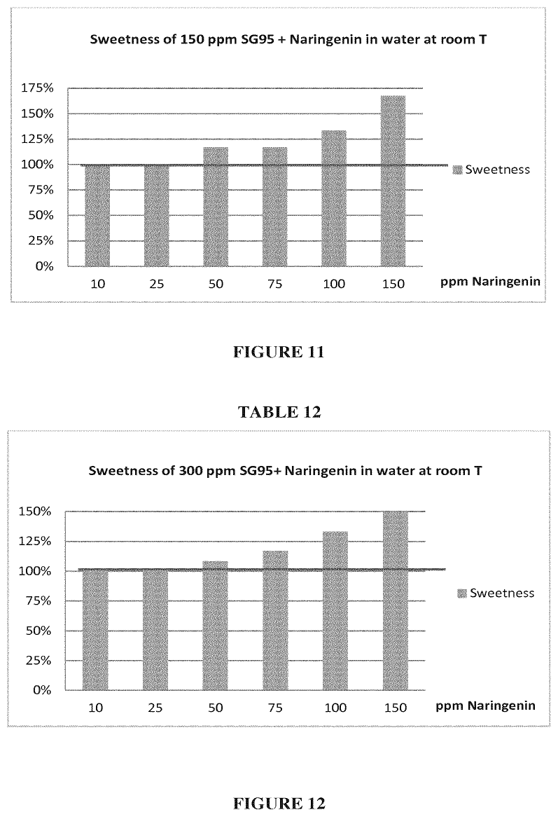

Example 11: Six samples were prepared and compared with 150 ppm SG95 (PureCircle) in water; 100% sweetness level (in red, FIG. 11). The first sample comprised 150 ppm SG95 with ppm Naringenin. The second sample was a 150 ppm SG95 with 25 ppm Naringenin. The third sample was a 150 ppm SG95 with 50 ppm Naringenin. The fourth sample comprised 150 ppm SG95 with 75 ppm Naringenin. The fifth sample was a 150 ppm SG95 with 100 ppm 10 Naringenin. The sixth sample was a 150 ppm SG95 with 150 ppm Naringenin. All samples were tested for sweetness intensity at room Temperature by four experts especially trained to evaluate sweet products. The data displayed in FIG. 11 shows that the addition of 10-25 ppm Naringenin to 150 ppm SG95 doesn't have any effect in sweetness. On the other hand, the addition from from 50 to 150 ppm Naringenin to 150 ppm SG95 solution has a sweetness enhancement effect.

Example 12: Six samples were prepared and compared with 300 ppm SG95 in water; 100% sweetness level (in red, FIG. 12). The first sample comprised 300 ppm SG95 with 10 ppm Naringenin. The second sample was a 300 ppm SG95 with 25 ppm Naringenin. The third sample was a 300 ppm SG95 with 50 ppm Naringenin. The fourth sample comprised 300 ppm SG95with 75 ppm Naringenin. The fifth sample was a 300 ppm SG95 with 100 ppm Naringenin. The sixth sample was a 300 ppm SG95 with 150 ppm Naringenin. All samples were tested for sweetness intensity at room Temperature. by four experts especially trained to evaluate sweet products. The data displayed in FIG. 12 shows that the addition of 10-25 ppm Naringenin to 300 ppm SG95 doesn't have any effect in sweetness. On the other hand, the addition from 50 to 150 ppm Naringenin to 300 ppm SG95 solution has a sweetness enhancement effect.

1. A method of enhancing the sweetness of sweetener in a food or beverage product comprising adding Naringenin to the product in an amount of 30 ppm to 200 ppm by weight of the total weight of the product wherein the Naringenin does not block the bitter taste of the product when compared to the beverage without Naringenin.

2. The method as recited in claim 1 wherein the sweetener is selected from the group consisting of “Sweeteners” include, but are not limited to the common saccharide sweeteners, e.g., sucrose, fructose (e.g., D-fructose), glucose (e.g., D-glucose), and sweetener compositions comprising natural sugars, such as corn syrup (including high fructose corn syrup) or other syrups or sweetener concentrates derived from natural fruit and vegetable sources, semisynthetic “sugar alcohol” sweeteners such as erythritol, isomalt, lactitol, mannitol, sorbitol, xylitol, maltodextrin, glycerol, threitol, arabitol, ribitol, dulcitol, and the like, and artificial sweeteners such as miraculin, aspartame, superaspartame, saccharin, saccharin-sodium salt, acesulfame-K,cyclamate, sodium cyclamate, and alitame. Sweeteners also include trehalose, melizitose, melibiose, raffinose, palatinose, lactulose, cyclamic acid, mogroside, tagatose (e.g., D-tagatose), maltose, galactose (e.g., D-galactose), L-rhamnose, D-sorbose, maunose (e.g., D-maunose), lactose, L-arabinose,D-ribose, D-glyceraldehyde, curculin, brazzein, mogroside, Neohesperidin dihydrochalcone (NHDC), neotame and other aspartame derivatives, D-tryptophan, D-leucine, D-threonine, glycine, D-asparagine, D-phenylalanine, L-proline, maltitol, hydrogenated glucose syrup (HGS), magap, sucralose, lugduname, sucrononate, sucrooctate, monatin, phyllodulcin, hydrogenated starch hydrolyzate (HSH), stevioside, rehaudioside A and other sweet Stevia based glycosides, lo han guo, thaumatin, monellin, carrelameand other guanidine-based sweeteners.

3. The method as recited in claim 1 or 2 wherein the product or beverage is not coffee.

4. A food or beverage product comprising Naringenin in an amount of from 30 to 200 ppm, by weight, of the total weight of the product, and a sweetener wherein the Naringenin does not block the bitter taste of the product.

5. A food or beverage product comprising Naringenin in an amount of from 30 to 200 ppm, by weight, of the total weight of the product, and a sweetener wherein the product is not a product selected from coffee, tea, a cosmetic and a pharmaceutical.

6. The food or beverage as recited in claim 4 wherein the sweetener is selected from the group consisting of “Sweeteners” include, but are not limited to the common saccharide sweeteners, e.g., sucrose, fructose (e.g., D-fructose), glucose (e.g., D-glucose), and sweetener compositions comprising natural sugars, such as corn syrup (including high fructose corn syrup) or other syrups or sweetener concentrates derived from natural fruit and vegetable sources, semisynthetic “sugar alcohol” sweeteners such as erythritol, isomalt, lactitol, mannitol, sorbitol, xylitol, maltodextrin, glycerol, threitol, arabitol, ribitol, dulcitol, and the like, and artificial sweeteners such as miraculin, aspartame, superaspartame, saccharin, saccharin-sodium salt, acesulfame-K,cyclamate, sodium cyclamate, and alitame. Sweeteners also include trehalose, melizitose, melibiose, raffinose, palatinose, lactulose, cyclamic acid, mogroside, tagatose (e.g., D-tagatose), maltose, galactose (e.g., D-galactose), L-rhamnose, D-sorbose, maunose (e.g., D-maunose), lactose, L-arabinose,D-ribose, D-glyceraldehyde, curculin, brazzein, mogroside, Neohesperidin dihydrochalcone (NHDC), neotame and other aspartame derivatives, D-tryptophan, D-leucine, D-threonine, glycine, D-asparagine, D-phenylalanine, L-proline, maltitol, hydrogenated glucose syrup (HGS), magap, sucralose, lugduname, sucrononate, sucrooctate, monatin, phyllodulcin, hydrogenated starch hydrolyzate (HSH), stevioside, rebaudioside A and other sweet Stevia based glycosides, lo han guo, thaumatin, monellin, carrelameand other guanidine-based sweeteners.

7. The food or beverage as recited in any one of claims 4 to 6 wherein the product or beverage is not coffee.
